SEQUENCE LISTING (1 sequence)

SEQ ID NO: 1 - protein, 303 aa, Escherichia coli
MKITVLGCGALGQLWLTALCKQGHEVQGWLRVPQPYCSVNLVETDGSIFNESLTANDPDFLATSDLLLVTLKAWQVSDAVKSLASTLPVTTPILLIHNGMGTIEELQNIQQPLLMGTTTHAARRDGNVIIHVANGITHIGPARQQDGDYSYLADILQTVLPDVAWHNNIRAELWRKLAVNCVINPLTAIWNCPNGELRHHPQEIMQICEEVAAVIEREGHHTSAEDLRDYVMQVIDATAENISSMLQDIRALRHTEIDYINGFLLRRARAHGIAVPENTRLFEMVKRKESEYERIGTGLPRPW

(19) United States
(12) Patent Application Publication
(10) Pub. No.: US 2025/0154543 A1
(43) Pub. Date: May 15, 2025

(54) REDUCED PANTOTHENIC ACID LEVELS IN FERMENTATIVE PRODUCTION OF OLIGOSACCHARIDES

(71) Applicant: Chr. Hansen A/S, Hoersholm (DK)

(72) Inventors: Henning FRERIGMANN, Rheinbreitbach (DE); Katja PARSCHAT, Rheinbreitbach (DE); Markus ENGLERT, Rheinbreitbach (DE); Christian TROETSCHEL, Rheinbreitbach (DE)

(73) Assignee: Chr. Hansen A/S, Hoersholm (DK)

(21) Appl. No.: 18/722,142

(22) PCT Filed: Dec. 15, 2022
(86) PCT No.: PCT/EP2022/086205

§ 371 Date: Jun. 20, 2024

(30) Foreign Application Priority Data
Dec. 22, 2021 (EP) EP21217103

(51) Int. Cl. C12P 19/04; C12N 9/04

(52) U.S. Cl. CPC C12P 19/04; C12N 9/0006; C12Y 101/01169

(57) ABSTRACT

Disclosed are means and methods for the fermentative production of an oligosaccharide of interest by a genetically engineered microbial cell, wherein concomitant biosynthesis of pantothenic acid is reduced or abolished.

The present invention relates to the fermentative production of an oligosaccharide of interest, and to genetically engineered microbial cells for the production of the oligosaccharide of interest. More specifically, the present invention concerns the fermentative production of an oligosaccharide of interest, wherein said oligosaccharide is suitable for use in an infant formula.

### BACKGROUND

Breastfeeding, also called nursing, is the process of feeding a mother's breast milk to an infant, regardless of whether the milk is fed directly from the breast or collected from the breast and subsequently bottle-fed to the infant. Breastfeeding is widely advocated as “ideal” for babies and infants due to the health benefits of breast milk. Health organizations, including the World Health Organization (WHO), recommend breastfeeding exclusively for at least the first six months of the baby's life. Despite this recommendation, less than 40% of infants worldwide are exclusively breastfed at an age younger than half a year. Instead, these infants are fed manufactured food, called infant formula (IF), usually prepared for bottle-feeding from powder or as ready-to-use liquid preparation. The U.S. Federal Food, Drug, and Cosmetic Act (FFDCA) defines infant formula as “a food which purports to be or is represented for special dietary use solely as a food for infants by reason of its simulation of human milk or its suitability as a complete or partial substitute for human milk”.

In 2001 a WHO report found that infant formula prepared in accordance with applicable Codex Alimentarius standards is a safe complementary food and a suitable breast milk substitute. In 2003, the WHO and the United Nations Children's Fund (UNICEF) published their Global Strategy for Infant and Young Child Feeding, which restated that “processed-food products for young children should, when sold or otherwise distributed, meet applicable standards recommended by the Codex Alimentarius Commission”, and also warned that “lack of breastfeeding—and especially lack of exclusive breastfeeding—during the first half-year of life possesses an important risk factor for infant and childhood morbidity and mortality”. In this sense, infant formula is one of the most regulated food in the world.

The nutritional value of infant formula as well as their content of ingredients including nutrients, vitamins and minerals is strictly prescribed. For example, the nutrient content of infant formula for sale in the United States is regulated by the Food and Drug Administration (FDA) based on recommendations by the American Academy of Pediatrics Committee on Nutrition. Addition of prebiotic oligosaccharides such as galactooligosaccharides (GOS), fructooligosaccharides (FOS) and human milk oligosaccharides (HMOs) to infant formula has been allowed several years ago.

Prebiotic oligosaccharides are non-digestible carbohydrates, i.e. carbohydrates that cannot be metabolized by humans. However, prebiotic carbohydrates can be metabolized by specific non-pathogenic intestinal bacteria. Promoting growth of these intestinal bacteria by supplying prebiotic oligosaccharides aides in preventing bacterial infections by out-competing the pathogens for nutrients and by impairing their growth due to secretion of acidic metabolites by said non-pathogenic bacteria, thereby lowering the pH in the gut. Thus, prebiotics are added to infant formula to enhance the development of a “healthy” gut microbiome in the infants.

GOS and FOS are not naturally occurring prebiotic carbohydrates and designate families of oligosaccharides which possesses a limited complexity. However, a much more diverse and complex family of prebiotic oligosaccharides is found in human milk. This family of oligosaccharides is designated as human milk oligosaccharides (HMOs), and the family of HMOs appear more advantageous to the well-being infants than the artificial surrogates.

HMOs are exclusively found in human milk and represent the third most abundant solid fraction of human milk. HMOs have prebiotic effects, thus shaping the gut microbiome by promoting growth of beneficial bacteria, e. g. Bifidobacteria and Lactobacilli, and suppress growth of pathogenic bacteria, they inhibit binding of bacterial and viral pathogens to epithelial cells, they support the development of an infant's immune system, and they enhance brain development and cognitive capabilities. Besides their beneficial effects on an infant's development, HMOs are also assumed to improve the gut microbiome and health of adults.

More than 150 structurally distinct HMOs have been identified so far. All of them are composed of no more than five different monosaccharide moieties, namely glucose, galactose, L-fucose, N-acetylglucosamine, and N-acetyl neuraminic acid (sialic acid). HMOs are segregated into neutral HMOs and acidic HMOs based on their chemical composition. Members of the latter subset of HMOs contain at least one sialic acid (i.e. N-acetylneuraminic acid) moiety, and are therefore also called “sialylated HMOs”. Neutral HMOs may or may not contain a fucose moiety.

Milk from some dairy animals contains oligosaccharides possessing structural equivalence to HMOs. However, the oligosaccharide composition of dairy milk is less complex than that of human milk, and the amount of oligosaccharides in dairy milk is much lower than in human milk.

Isolation of specific oligosaccharides from milk, especially from milk of a dairy animal, in large amounts seems not feasible to date. Oligosaccharides can be synthesized chemically or by in vitro biocatalysis. However, chemical synthesis of oligosaccharides involves stepwise addition of monosaccharide moieties at the reducing or non-reducing end of an existing saccharide chain, which addition includes adding protective groups to and removing them from side chains within the monosaccharide moieties of the saccharide chain. Considering the peculiarities of stereochemistry, it turned out that chemical synthesis is also not feasible for providing oligosaccharides in an industrial scale at reasonable costs and at sufficiently sustainable conditions to be used in the food industry. Alternatively, oligosaccharides may be produced by biochemical reactions in vitro, i.e. by biocatalysis, wherein enzymes catalyze the formation of glycosidic bonds between saccharide moieties. However, biocatalysis is also not feasible for an industrial scale manufacturing of oligosaccharides at reasonable costs due to expensive educts that are required, e. g. nucleotide-activated monosaccharides or complex oligosaccharides.

To overcome above outlined drawbacks in the production of HMOs and for providing HMOs in an industrial scale at reasonable costs such that HMOs can become reasonably priced for being usable in infant formula, fermentative production processes using metabolically engineered bacterial cells, e.g., Escherichia coli, were developed. Such in vivo production approaches overcome the need of using expensive educts, because the metabolically engineered bacteria are able to synthesize said educts and the desired HMO intracellularly. The desired HMO can then be purified from the bacteria or from the fermentation broth. Such in vivo production processes proved to provide HMOs in sufficiently pure form and at economically feasible costs for their use as ingredients in infant formula.

Referring to the prescribed nutrient content of infant formula for sale in the United States mentioned herein before, the following compounds must be included in all infant formula produced in the U.S.: protein, fat, linoleic acid, vitamins A, C, D, E, K, thiamin, riboflavin, vitamins B₆, and B₁₂, niacin, folic acid, pantothenic acid, calcium, magnesium, iron, zinc, manganese, copper, phosphorous, iodine, sodium chloride, potassium chloride, carbohydrates, and nucleotides. In this respect, the US FDA issued nutrient specifications in regulation 21 CRF 107.100 defining a minimum concentration of pantothenic acid of at least 0.3 mg/100 kcal. Infant formulae currently contain pantothenic acid in concentrations of about 0.5 to about 0.9 mg/100 kcal.

According to EU regulation No. 609/2013 and its supplemental commission delegated regulation (EU) 2016/127 dated Sep. 25, 2015, the concentration of pantothenic acid in an infant formula shall not be less than 0.4 mg/100 kcal and shall not exceed 2 mg/100 kcal when prepared for consumption as indicated by the manufacturer.

Pantothenate (vitamin B5), the salt of pantothenic acid (both terms are used interchangeably in the instant disclosure unless stated otherwise), is the key precursor for the biosynthesis of coenzyme A (CoA) and carrier proteins that have a phosphopantetheine prosthetic group. Coenzyme A is an essential cofactor for cell growth, it is involved in many metabolic reactions, including the synthesis of phospholipids, synthesis and degradation of fatty acids, and the operation of the tricarboxylic acid cycle.

Most prokaryotes, fungi, and plants are able to synthesize pantothenate de novo, whilst animals rely on exogenous vitamin B5 supply. In bacteria such as E. coli biosynthesis of pantothenic acid comprises several steps (FIG. 1). Alpha-ketoisovalerate is converted to α-ketopantoate by the ketopantoate hydroxymethyltransferase (PanB; UniProtKB accession no. P31057 for E. coli K12 PanB). Then, α-ketopantoate is reduced to pantoate by the ketopantoate reductase PanE (UniProtKB accession no. P0A9J4 for E. coli K12 PanE). The pantothenate synthetase (PanC; UniProtKB accession no. P31663 for E. coli K12 PanC) catalyzes the condensation of pantoate and β-alanine to pantothenate. Beta-alanine is produced from aspartate by the aspartate α-decarboxylase (PanD; UniProtKB accession no. P0A790 for E. coli K12 PanD). The genes encoding PanB and PanC form an operon panBC in E. coli, while the panD gene is located downstream of that operon separated by one gene only. PanE encoding the NADPH dependent ketopantoate reductase is located in another part of the E. coli genome and forms an operon with yajL that is not involved in pantothenate synthesis.

Pantothenate is further converted to coenzyme A by several enzymatic reactions. However, bacteria also secrete pantothenate into their environment as production of pantothenic acid in E. coli is several fold higher than its intracellular demand and thus is found to accumulate in the fermentation medium (Jackowski, S. and Rock, C. O. (1984) J. Bacteriol. 158, 115-120). E. coli is also able to internalize exogenous pantothenic acid, and internalization of pantothenic acid is mediated by their pantothenic acid permease PanF (UniProtKB accession no. P16256 for E. coli K12 PanF), which is a sodium/pantothenate symporter.

Pantothenic acid is a by-product found in the fermentation broth when culturing E. coli for HMO production. Given the prescribed maximum amounts of vitamin B5 in the infant formula it might be necessary to deplete pantothenic acid from the HMO preparation to render the HMO product suitable as ingredient for infant formula. This is particularly pertinent if the HMO concentrations within an infant formula should resemble the natural concentrations of HMOs in breast milk, namely between about 40 g to about 120 g HMOs per kg ready to use infant formula.

It may be possible to reduce the pantothenic acid level in a process stream obtained from a fermentation broth containing an HMO by crystallizing the HMO from the process stream. The HMO may be crystallized from water, an organic solvent, or a mixture of water and an organic solvent. Crystallization of the HMO can be improved by the addition of an organic acid to the HMO-containing solution.

Additionally, and/or alternatively, pantothenic acid can be separated from an oligosaccharide of interest by subjecting the process stream to at least one specific ion exchange treatment step for removal of pantothenic acid, namely an anionic ion exchange chromatography or adsorption of the pantothenic acid to an anion adsorption resin. The anion exchange resin or the anion adsorption resin may be a strong or a weak anion exchange resin, preferably it is a weak anion ion exchange resin.

Above outlined approaches for the removal of pantothenic acid require additional process steps in the purification scheme for producing an HMO by fermentative means. Moreover, using such specific ion exchange treatment step for removing pantothenic acid from a process stream might be feasible for purifying neutral HMOs. However, such ion exchange treatment step cannot be used for the purification and recovery of acidic sialylated HMOs such as sialyllactoses since acidic HMOs possess similar binding capabilities to the anion exchange resin to be used as pantothenic acid. This renders it virtually impossible to separate sialylated HMOs from pantothenic acid in an industrial scale using an ion exchange treatment for the removal of pantothenic acid.

To overcome aforesaid drawbacks, a process for the fermentative production of an oligosaccharide of interest has been developed, wherein the accumulation of pantothenic acid in the culture medium during oligosaccharide biosynthesis by a microbial cell is avoided or at least significantly reduced.

### SUMMARY

In a first aspect, provided are genetically engineered microbial cells for the production of an oligosaccharide of interest, wherein said genetically engineered microbial cells possess a metabolic pathway for the intracellular biosynthesis of the oligosaccharide of interest, and a low level of or no intracellular biosynthesis of pantothenic acid.

In a second aspect, provided is the use of a genetically engineered microbial cell possessing a metabolic pathway for the intracellular biosynthesis of an oligosaccharide of interest and a low level of or no intracellular biosynthesis of pantothenic acid for the production of the oligosaccharide of interest.

In a third aspect, provided is a method for the production of an oligosaccharide of interest, wherein the method comprises (i) providing a genetically engineered microbial cell which possesses a metabolic pathway for the intracellular biosynthesis of the oligosaccharide of interest, and a low level of or no intracellular biosynthesis of pantothenic acid; (ii) culturing the genetically engineered microbial in a medium and at conditions that are permissive for the microbial cell to synthesize the oligosaccharide of interest intracellularly; and optionally (iii) retrieving the oligosaccharide of interest.

In a fourth aspect, provided is a preparation of an oligosaccharide of interest, wherein said preparation is obtained by a method wherein a genetically engineered microbial cell which possesses a metabolic pathway for the intracellular biosynthesis of the oligosaccharide of interest, and a low level of or no intracellular biosynthesis of pantothenic acid, has been cultured in a medium and at conditions that are permissive for the microbial cell to synthesize the oligosaccharide of interest intracellularly, and wherein said preparation of the oligosaccharide contains a low level of pantothenic acid or no pantothenic acid.

In a fifth aspect, provided is the use of the preparation of the oligosaccharide of interest for the manufacturing of a nutritional composition.

In a sixth aspect, provided is a nutritional composition containing a preparation of an oligosaccharide of interest, which preparation contains a low level of pantothenic acid or no pantothenic acid.

### BRIEF DESCRIPTION OF THE DRAWINGS

FIG. 1 illustrates the metabolic pathway for the intracellular biosynthesis of pantothenic acid in E. coli as shown by Leonardi, R. and Jackowski, S., Biosynthesis of Pantothenic Acid and Coenzyme A; EcoSal Plus. 2007 April; 2(2):10.1128/ecosalplus.3.6.3.4.

FIG. 2 displays the amino acid sequence of the Escherichia coli K-12 ketopantoate reductase PanE (UniProtKB Entry number P0A9J4, UniProt release 2021_03 as published Jun. 2, 2021).

FIG. 3 shows two graphs illustrating the growth of two different E. coli strains producing 3′-sialyllactose and the amounts of pantothenic acid in their culture media, wherein FIG. 3B displays the growth and the pantothenic acid level in the culture medium of an E. coli strain that was genetically engineered to possess a low level of intracellular pantothenic acid biosynthesis, while FIG. 3A displays the growth and the pantothenic acid level in the culture medium of its progenitor strain prior to being genetically engineered for having its pantothenic acid biosynthesis reduced.

FIG. 4 shows two graphs illustrating the growth of two different E. coli strains producing lacto-N-tetraose and the amounts of pantothenic acid in their culture media, wherein FIG. 4B displays the growth and the pantothenic acid level in the culture medium of an E. coli strain that was genetically engineered to possess a low level of intracellular pantothenic acid biosynthesis, while FIG. 4A displays the growth and the pantothenic acid level in the culture medium of its progenitor strain prior to being genetically engineered for having its pantothenic acid biosynthesis reduced.

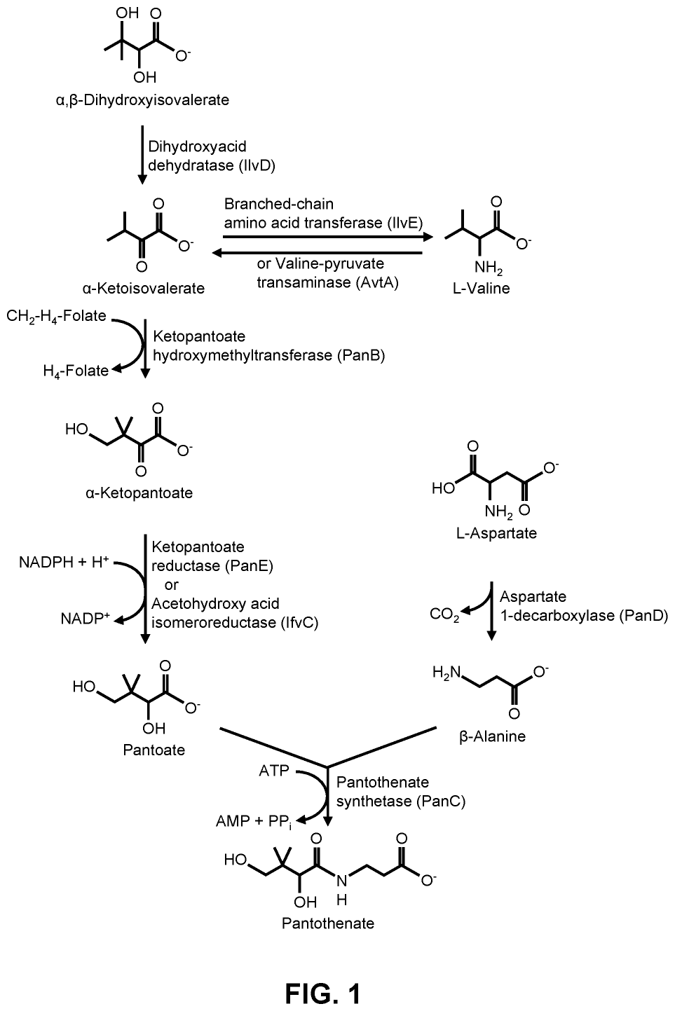

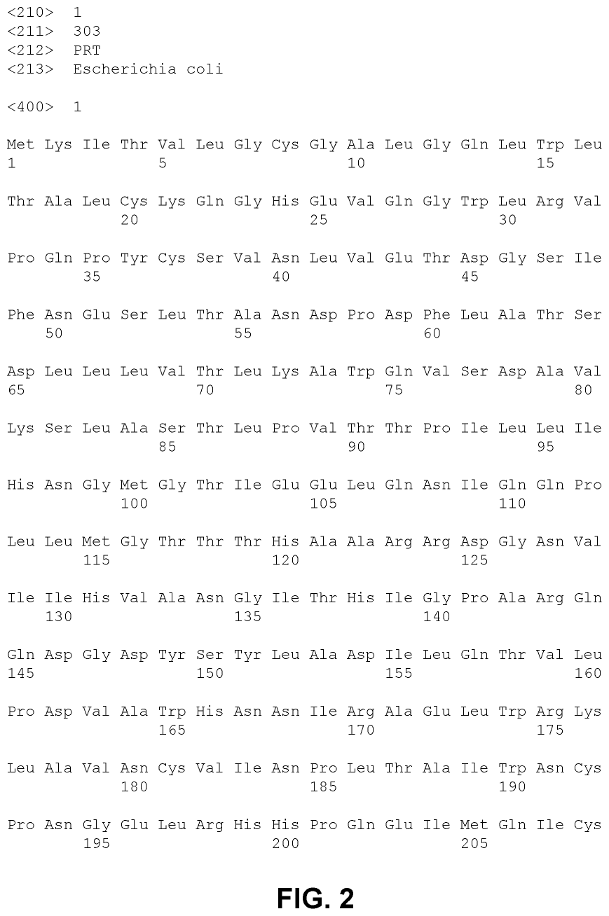

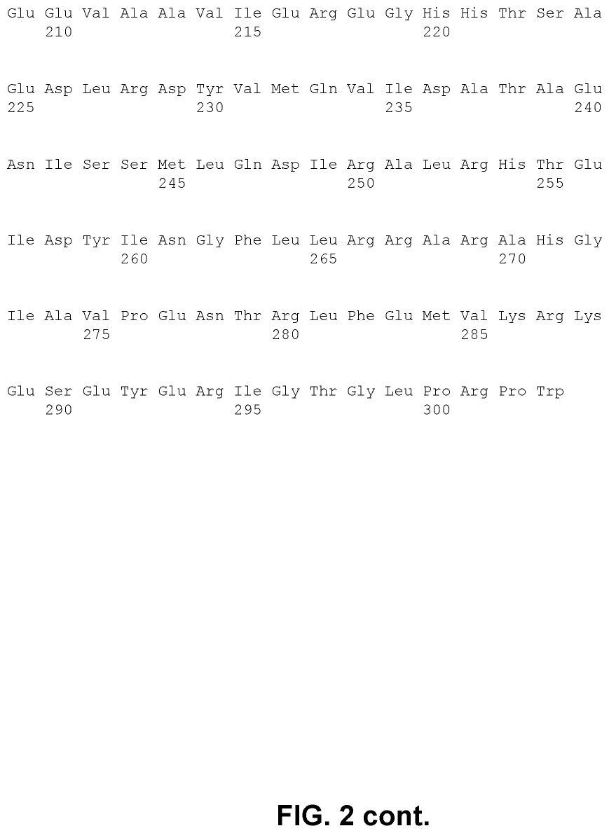

### DETAILED DESCRIPTION

According to the first aspect, provided is a genetically engineered microbial cell for the production of an oligosaccharide of interest, wherein said microbial cell possess a metabolic pathway for the intracellular biosynthesis of the oligosaccharide of interest, and wherein the genetically engineered microbial cells also possess a low level of intracellular biosynthesis of pantothenic acid or possess no intracellular biosynthesis of pantothenic acid.

The genetically engineered microbial cell is a microbial cell that has been genetically engineered to possess the metabolic pathway for the intracellular biosynthesis of the oligosaccharide of interest and/or to possess the low level of pantothenic acid biosynthesis or to be incapable of pantothenic acid biosynthesis.

The term “microbial cell” as used herein refers to unicellular organisms. The microbial cell may be a prokaryotic cell or a eukaryotic cell. Suitable procaryotic cells include bacterial cells and archaebacterial cells. Suitable eukaryotic cells include yeast cells and fungal cells.

In an additional and/or alternative embodiment, the prokaryotic cell is a bacterial cell, preferably a bacterium of a genus selected from the group consisting of Bacillus, Bifidobacterium, Clostridium, Corynebacterium, Enterococcus, Lactobacillus, Lactococcus, Micrococcus, Micromonospora, Pseudomonas, Rhodococcus and Sporolactobacillus. Suitable bacterial species within said genera are Bacillus subtilis, B. licheniformis, B. coagulans, B. thermophilus, B. laterosporus, B. megaterium, B. mycoides, B. pumilus, B. lentus, B. cereus, B. circulans, Bifidobacterium longum, B. infantis, B. bifidum, Citrobacter freundii, Clostridium cellulolyticum, C. ljungdahlii, C. autoethanogenum, C. acetobutylicum, Corynebacterium glutamicum, Enterococcus faecium, E. thermophiles, Escherichia coli, Erwinia herbicola (Pantoea agglomerans), Lactobacillus acidophilus, L. salivarius, L. plantarum, L. helveticus, L. delbrueckii, L. rhamnosus, L. bulgaricus, L. crispatus, L. gasseri, L. casei, L. reuteri, L. jensenii, L. lactis, Pantoea citrea, Pectobacterium carotovorum, Proprionibacterium freudenreichii, Pseudomonas fluorescens, P. aeruginosa, Streptococcus thermophiles and Xanthomonas campestris.

In some embodiments, the eukaryotic cell is a yeast cell, preferably a yeast cell selected from a genus of the group consisting of Saccharomyces sp., Saccharomycopsis sp., Pichia sp., Hansenula sp., Kluyveromyces sp., Yarrowia sp., Rhodotorula sp., and Schizosaccharomyces sp. In additional and/or alternative embodiments, the yeast cell is a Saccharomyces cerevisiae cell, a Pichia pastoris cell, or a Hansenula polymorpha cell.

The genetically engineered microbial cell is a microbial cell for the production of an oligosaccharide of interest.

As used herein, the term “oligosaccharide” typically refers to a polymeric saccharide molecule consisting of at least three monosaccharide moieties, but of no more than 12 monosaccharide moieties, preferably of no more than 10 monosaccharide moieties. The monosaccharide moieties of an oligosaccharide are linked to one another by glycosidic bonds. An oligosaccharide may consist of a linear chain of monosaccharide moieties, or the oligosaccharide may constitute a branched chain of monosaccharide moieties, wherein at least one monosaccharide moiety has at least three monosaccharide moieties bound to it by glycosidic bonds. The monosaccharide moieties of an oligosaccharide can be selected from the group consisting of aldoses (e.g. arabinose, xylose, ribose, desoxyribose, lyxose, glucose, idose, galactose, talose, allose, altrose, mannose), ketoses (e.g. ribulose, xylulose, fructose, sorbose, tagatose), deoxysugars (e.g. rhamnose, fucose, quinovose), deoxy-aminosugars (e.g. N-acetylglucosamine, N-acetyl-mannosamine, N-acetylgalactosamine), uronic acids (e.g. galacturonsäure, glucuronsäure) and ketoaldonic acids (e.g. N-acetylneuraminic acid).

The expression “oligosaccharide of interest” as used herein refers to the oligosaccharide that is supposed to be synthesized by the microbial cell. Typically, the microbial cell has been selected and/or genetically engineered to synthesize a particular oligosaccharide, i.e. the oligosaccharide of interest. Any other oligosaccharide that may be synthesized by the microbial cell than the oligosaccharide of interest is considers to be a by-product, regardless of whether said other oligosaccharide than the oligosaccharide of interest is a reaction product of an intermediate reaction in the biosynthesis of the oligosaccharide of interest or constitutes an endproduct of another metabolic pathway than the metabolic pathway for the biosynthesis of the oligosaccharide of interest.

In some embodiments, the oligosaccharide of interest is a human milk oligosaccharide, i.e. an oligosaccharide selected from the group of oligosaccharides (HMOs) that are present in human milk. Human Milk Oligosaccharides constitute a diverse mixture of non-digestible oligosaccharides that are present in human milk. Human milk is unique with respect to its oligosaccharide composition and quantities. To date, more than 150 structurally distinct HMOs have been identified. The vast majority of HMOs are characterized by a lactose moiety (Gal-β1,4-Glc) at their reducing end. Many HMOs contain a fucose moiety and/or a sialic acid moiety. More generally speaking, the monosaccharides from which HMOs are derived are D-glucose, D-galactose, N-acetylglucosamine, L-fucose and/or N-acetylneuraminic acid.

In an additional and/or alternative embodiment, the oligosaccharide of interest is a HMO selected from the group consisting of 2′-fucosyllactose (2′-FL), 3-fucosyllactose (3-FL), 2′,3-difucosyllactose (DFL), lacto-N-triose II, lacto-N-tetraose (LNT), lacto-N-neotetraose (LNnT), lacto-N-fucopentaose I (LNFP-I), lacto-N-neofucopentaose I (LNnFP-I), lacto-N-fucopentaose II (LNFP-II), lacto-N-fucopentaose III (LNFP-III), lacto-N-fucopentaose V (LNFP-V), lacto-N-neofucopentaose V (LNnFP-V), lacto-N-hexaose (LNH), lacto-N-neohexaose (LNnH), para-lacto-N-hexaose (paraLNH), para-lacto-N-neohexaose (paraLNnH), difucosyl-lacto-N-neohexaose (DF-LNnH), lacto-N-difucosylhexaose I, lacto-N-difucosylhexaose II, para-lacto-N-fucosylhexaose (paraLNH), fucosyl-lacto-N-sialylpentaose a (F-LST-a), fucosyllacto-N-sialylpentaose b (F-LST-b), fucosyl-lacto-N-sialylpentaose c (F-LST-c), fucosyl-lacto-N-sialylpentaose c, disialyl-lacto-N-fucopentaose, 3-fucosyl-3′-sialyllactose (3F-3′-SL), 3-fucosyl-6′-sialyllactose (3F-6′-SL), lacto-N-neodifucohexaose I, 3′-sialyllactose (3-SL), 6′-sialyllactose (6-SL), sialyllacto-N-tetraose a (LST-a), sialyllacto-N-tetraose b (LST-b), sialyllacto-N-tetraose c (LST-c), disialyllacto-N-tetraose (DS-LNT), Disialyl-lacto-N-fucopentaose (DS-LNFP V), lacto-N-neodifucohxaose I (LNnDFH I), 3′-galactosyllactose (3′-GL), 6′-galactosyllactose (6′-GL).

The genetically engineered microbial cell is genetically modified for the production of an oligosaccharide of interest. Typically the oligosaccharide of interest is an oligosaccharide that is not naturally synthesized by the microbial cell. Thus, the microbial cell has usually been genetically engineered to be able to intracellularly synthase the oligosaccharide of interest, i.e. the microbial cell has been genetically engineered to synthesize the oligosaccharide of interest intracellularly if cultured in a medium and at conditions that are permissive for the synthesis of the oligosaccharide of interest. Hence, the genetically engineered microbial cell has been genetically engineered to possess a metabolic pathway for the intracellular biosynthesis of the oligosaccharide of interest.

The genetically engineered microbial cell possesses a metabolic pathway for the intracellular biosynthesis of the oligosaccharide of interest. Thus, the genetically engineered microbial cell possesses all enzymes and transporters that are necessary to provide an acceptor saccharide for acquiring a monosaccharide moiety, to provide a donor substrate for the monosaccharide moiety which is to be transferred to the acceptor saccharide, and to catalyze the conversion of the acceptor saccharide to the oligosaccharide of interest by transferring the monosaccharide moiety from the donor substrate to the acceptor saccharide.

In some embodiments, the acceptor saccharide is a disaccharide that is converted into a trisaccharide. In other embodiments, the acceptor saccharide is a trisaccharide that is converted into a tetrasaccharide. In other embodiments, the acceptor saccharide is a tetrasaccharide that is converted into a pentasaccharide. In some embodiments, the acceptor saccharide is a pentasaccharide that is converted into a hexasaccharide. The acceptor saccharide may be internalized by the microbial cell, preferably by utilizing a specific transporter that is present in the microbial cell's cell membrane. Additionally and/or alternatively the acceptor saccharide can be synthesized by the microbial cell intracellularly.

In some embodiments, the acceptor saccharide is synthesized intracellularly by the microbial cell from a monosaccharide that has been internalized by the microbial cell and is elongated by a monosaccharide moiety to obtain a disaccharide, wherein said disaccharide may be further elongated by the addition of further monosaccharide moieties such that and oligosaccharide is synthesized intracellularly which consists of three, four, five, six or more monosaccharide moieties, and which constitutes the acceptor saccharide for the biosynthesis of the oligosaccharide of interest. For example, the microbial cell internalizes glucose which is converted to lactose by the addition of a galactose moiety. Said lactose may then be converted to an oligosaccharide such as, e.g., 2′-FL, 3-FL, LNT-II, LNT, LNnT, 3′-SL, 6′-SL. Each of said oligosaccharides may be an oligosaccharide of interest or may constitute an acceptor saccharide.

In some embodiments, the microbial cell has been genetically engineered to possess the enzymes which catalyze the conversion of the acceptor saccharide, i.e. a disaccharide or an oligosaccharide, to the oligosaccharide of interest by transferring the monosaccharide moiety from the donor substrate to the acceptor saccharide. Hence, the microbial cell may contain and express at least one recombinant gene which encodes an enzyme catalyzing the conversion of the acceptor saccharide to the oligosaccharide of interest.

Genes or equivalent functional nucleotide sequences encoding the enzymatic ability to the microbial cell to add a monosaccharide moiety from the donor substrate to an acceptor saccharide may be a homologous nucleotide sequence or a heterologous nucleotide sequence. The term “homologous” as used herein with respect to nucleotide sequences refers to nucleotide sequences that are native to the species the microbial cell for producing the oligosaccharide of interest belongs to. The term “heterologous” as used herein with respect to nucleotide sequences refers to nucleotide sequences that are artificially generated or derived from a different species than the microbial cell for producing the oligosaccharide of interest belongs to. Heterologous nucleotide sequences that are derived from a different species than the one of the microbial cell for producing the oligosaccharide of interest may originate from a plant, an animal including humans, a bacterium, an archaea, a fungus or a virus.

The enzyme catalyzing the transfer a monosaccharide moiety to the acceptor molecule is a glycosyltransferase or a transglycosidase. In some embodiments the glycosyltransferase catalyzes the transfer of a monosaccharide moiety from a nucleotide-activated sugar as donor substrate to the acceptor saccharide. The glycosyltransferase may be selected from the group consisting of galactosyltransferases, glucosamyltransferases, sialyltransferases, N-acetylglucosaminyltransferases, N-acetylgalactosaminyltransferases, glucuronosyltransferases, mannosyltransferases, xylosyltransferases, and fucosyltransferases. In an additional and/or alternative embodiment, the glycosyltransferase is selected from the group consisting of α-1,2-mannosyltransferases, β-1,4-xylosyltransferases, β-1,3-N-acetylglucosaminyltransferases, β-1,6-N-acetylglucosaminyltransferases, β-1,3-N-acetylgalactosaminyltransferases, β-1,4-N-acetylgalactosaminyltransferases, α-1,3-N-acetylgalactosaminyltransferases, β-1,3-galactosyltransferases, β-1,4-galactosyltransferases, β-1,6-galactosyltransferases, α-1,3-glucosyltransferases, α-1,4-glucosyltransferases, α-2,3-sialyltransferases, α-2,6-sialyltansferases, α-2,8-sialyltansferases, α-1,2-fucosyltransferases and α-1,3/1,4-fucosyltransferases.

It is understood that a fucosyltransferase uses GDP-L-fucose as donor substrate for the transfer of the fucose moiety to the acceptor saccharide, while a sialyltransferase uses CMP-N-acetylneuraminic acid as donor substrate for the transfer of N-acetylneuraminic acid to the acceptor saccharide, and so on.

The nucleotide-activated sugar may be synthesized by the genetically engineered microbial cell in a de novo pathway. Additionally and/or alternatively, the microbial cell may use a salvage pathway for providing the nucleotide-activated sugar.

In those embodiments wherein the donor substrate is synthesized by a de novo pathway, the microbial cell possesses the enzymes that are necessary for the de novo biosynthesis of the nucleotide-activated sugar. In some embodiments, the microbial cell has been genetically engineered to possess at least one recombinant gene encoding an enzyme that is necessary for the de novo biosynthesis pathway for the nucleotide-activated sugar. In the de novo biosynthesis pathway, the nucleotide-activated sugar is synthesized from a “simple” carbon source such as glucose, sucrose, fructose, glycerol or the like.

The at least one gene encoding an enzyme that is necessary for the de novo biosynthesis pathway of the nucleotide-activated sugar may be endogenous to the microbial cell or they can be introduced from exogenous sources into the microbial cell to be expressed. Expression, i.e. functional transcription and translation, of proteins encoded by endogenous genes can be modified by genetically engineering the microbial cell. For example, alteration of the expression of an endogenous gene may be achieved by modifying the gene's transcriptional promotor, by modifying the gene's ribosome binding site and/or by modifying the codon usage of the gene's protein-coding nucleotide sequence. In addition, the protein-coding nucleotide sequence of the gene may be modified such that the activity and/or the specificity of the enzyme being encoded by said gene becomes favorable for the desired biosynthesis pathway. A person skilled in the art would know which gene(s) need to be expressed in the genetically modified organism for de novo synthesis of nucleotide-activated sugar donor molecules.

In embodiments wherein a salvage pathway is used for the biosynthesis of the nucleotide-activated sugar as donor substrate, the microbial cell comprises an enzyme which catalyzes the linkage of a nucleotide and a monosaccharide. An example of such an enzyme is the bifunctional fucokinase/L-fucose-1-phosphateguanylyltransferase FKP from Bacteroides fragilis which catalyzes a kinase reaction and a pyrophosphatase reaction to form GDP-L-fucose. A further example of an enzyme which catalyzes the linkage of a nucleotide and a monosaccharide is the N-acetylneuraminate cytidyltransferase NeuA from Neisseria meningitidis which forms CMP-neuraminic acid.

In some embodiment, the microbial cell possessing a salvage pathway for providing a nucleotide-activated sugar as donor substrate contains a recombinant gene encoding the enzyme which catalyzes the linkage of the nucleotide and the monosaccharide for the expression of said gene.

A monosaccharide that is used as substrate for the formation of the nucleotide-activated sugar in the salvage pathway may be internalized by the microbial cell utilizing a monosaccharide import protein. Examples of such monosaccharide import proteins are a fucose permease, e.g. FucP, and a sialic acid importer, e.g. NanT from E. coli. The monosaccharide import proteins may be encoded by and expressed from an endogenous gene or may be encoded by and expressed from a recombinant gene.

In certain embodiments, at least one endogenous gene of the microbial cell which encodes an enzyme or a protein that prevents or interferes with the biosynthesis of the nucleotide-activated sugar and/or the oligosaccharide of interest is deleted or functionally inactivated.

In some embodiments, the genetically engineered microbial cell possesses a saccharide transporter in its cell membrane. The saccharide transporter translocates the oligosaccharide of interest from the cytoplasm of the microbial cell across its cell membrane into the culture medium or—in case of gram-negative bacteria—into the periplasmic space.

A saccharide transporter mediating the translocation of a carbohydrate, e.g. the oligosaccharide of interest, across the cell membrane may consist of a single polypeptide or may consist of multiple polypeptides (each one considered being a subunit) which forming a homomeric or heteromeric complex that translocates the carbohydrate across the cell membrane.

In some embodiments, the saccharide transporter is a member selected from the group consisting of the major facilitator superfamily (MFS), the sugar:cation symporter family, the nucleoside-specific transporter family, the ATP-binding cassette superfamily, and the phosphotransferase system family.

In certain embodiments, the saccharide transporter is encoded by and expressed from a functional recombinant gene. Hence, in such embodiments, the microbial cell comprises a functional gene or an operon which encodes the saccharide transporter or at least one subunit of a saccharide transporter complex which facilitates the translocation of the intracellularly produced oligosaccharide of interest across the inner membrane of the bacterial cell into its periplasm.

The term “operon” as used herein refers to a nucleotide sequence comprising two or more protein coding sequences that are transcribed from the same regulatory element for mediating and/or controlling expression in the microbial cell.

The term “functional gene” as used herein refers to a nucleic acid molecule comprising a nucleotide sequence which encodes a protein or polypeptide, and which also contains regulatory sequences operably linked to said protein-coding nucleotide sequence (open reading frame) such that the nucleotide sequence which encodes the protein or polypeptide can be expressed in/by the cell bearing said functional gene. Thus, when cultured at conditions that are permissive for the expression of the functional gene, said gene is expressed, and the cell expressing said gene typically comprises the protein or polypeptide that is encoded by the protein coding region of the functional gene. As used herein, the terms “nucleic acid” and “polynucleotide” refer to a deoxyribonucleotide or ribonucleotide polymer in either single- or double-stranded form, and unless otherwise limited, encompasses known analogues of natural nucleotides that hybridize to nucleic acids in a manner similar to naturally occurring nucleotides.

The term “deregulated” as used herein with respect to endogenous genes refers to an altered expression of the protein coding region of said endogenous gene as compared to the expression of the gene in the native progenitor of the genetically engineered microbial cell. Expression of a gene can be deregulated by different means known to the skilled artisan. Examples of deregulating the expression of a gene include alterations of the native nucleotide sequence of the gene's promoter, alteration of the native nucleotide sequence of the gene's ribosomal binding site. The term “deregulated” comprises an increased expression of a gene as well as a decreased or impaired expression of the gene.

The term “operably linked” as used herein shall mean a functional linkage between a nucleotide sequence which controls expression (such as a promoter, signal sequence, or array of transcription factor binding sites) and a second nucleotide sequence, wherein the nucleotide sequence controlling expression effects transcription and/or translation of the second nucleotide sequence. Accordingly, the term “promoter” designates a nucleotide sequence which usually “precedes” a protein coding nucleotide sequence of a DNA polynucleotide and provides a site for initiation of the transcription into mRNA. “Regulator” DNA sequences, also usually “upstream” of (i.e., preceding) the protein-coding nucleotide sequence of a gene in a given DNA polymer, bind proteins that determine the frequency (or rate) of transcriptional initiation. Collectively referred to as “promoter/regulator” or “control” DNA sequence, these sequences which precede a selected gene (or series of genes) in a functional DNA polymer cooperate to determine whether the transcription (and eventual expression) of a gene will occur. Nucleotide sequences which “follow” a protein coding nucleotide sequence in a DNA polymer and provide a signal for termination of the transcription into mRNA are referred to as transcription “terminator” sequences.

The term “recombinant”, as used herein indicates that a polynucleotide, such as a gene or operon, has been created by means of genetic engineering. Hence, a recombinant gene, operon or polynucleotide is not naturally occurring in cells of the same species as the gram-negative bacterial cell for the production of the oligosaccharide of interest. The recombinant polynucleotide may contain a heterologous nucleotide sequence, or expresses a peptide or protein encoded by a heterologous nucleotide sequence (i.e., a nucleotide sequence that is foreign to said microbial cell). Recombinant microbial cells can contain genes that are not found in its native (non-recombinant) predecessor. Recombinant cells can also contain variants of genes that are found in the recombinant cell's native predecessor, wherein the genes were modified and re-introduced into the cell by technical means. The term “recombinant” also encompasses bacterial cells that contain a nucleotide sequence that is endogenous to the bacterial cell and has been modified without removing the nucleic acid molecule bearing said nucleotide sequence from the bacterial cell. Such modifications include those obtained by gene replacement, site-specific mutation, and related techniques. Accordingly, a “recombinant polypeptide” is one which has been produced by a recombinant cell.

A “heterologous nucleotide sequence” or a “heterologous nucleic acid”, as used herein, is one that originates from a source foreign to the particular host cell (e.g. from a different species), or, if from the same source, is modified from its original form. Thus, a heterologous nucleotide sequence may be a nucleotide sequence wherein a heterologous protein coding nucleotide sequence being operably linked to a promoter, wherein the protein coding nucleotide sequence and the nucleotide sequence of the promoter were obtained from different source organisms, or, if from the same source organism, either the protein-coding nucleotide sequence or the promoter has been modified from its original form. The heterologous nucleotide sequence may be stably introduced into the genome of the bacterial cell, e.g. by transposition, transfection, transformation, conjugation or transduction. The techniques that may be applied will depend on the bacterial cell and the specificities of the nucleic acid molecule to be introduced into the bacterial cell. Various techniques are known to a person skilled in the art and are, e.g., disclosed in Sambrook et al., Molecular Cloning: A Laboratory Manual, 2nd Ed., Cold Spring Harbor Laboratory Press, Cold Spring Harbor, N.Y. (1989). Accordingly, a “genetically engineered bacterial cell” or “genetically engineered microbial cell” is understood as a bacterial cell which has been transformed or transfected, or is capable of transformation or transfection by an exogenous polynucleotide sequence. Thus, the nucleotide sequences as used in the invention, may, e.g., be comprised in a vector which is to be stably transformed/transfected or otherwise introduced into host microorganism cells. A great variety of expression systems can be used to produce polypeptides. Such vectors include, among others, chromosomal, episomal and virus-derived vectors, e.g., vectors derived from bacterial plasmids, from bacteriophage, from transposons, from yeast episomes, from insertion elements, from yeast chromosomal elements, from viruses, and vectors derived from combinations thereof, such as those derived from plasmid and bacteriophage genetic elements, such as cosmids and phagemids. The expression system constructs may contain control regions that regulate as well as engender expression. Generally, any system or vector suitable to maintain, propagate or express polynucleotides and to synthesize a polypeptide in a bacterial host cell may be used for expression in this regard. The appropriate nucleotide sequence may be inserted into the expression system by any of a variety of well-known and routine techniques, such as, for example, those set forth in Sambrook et al., supra. Preferably, polynucleotides containing the recombinant nucleotide sequence are stably introduced into the genome of the microbial cell. Genomic integration can be achieved by recombination or transposition.

The functional gene or functional genes encoding the transporter or the transporter complex facilitating the transport of the oligosaccharide of interest across the inner membrane can either be endogenous or exogenous to the genetically engineered microbial cell.

In some embodiments the saccharide transporter gene is endogenous to the genetically engineered bacterial cell. Functional elements effecting synthesis of the protein that is encoded by the open reading frame of the saccharide transporter gene may have been modified such that the level of transcription of the open reading frame or translation of the mRNA leading to synthesis of the transporter protein is different from that naturally found in the predecessor cell. Modification of the functional elements can either increase the transcription and/or translation level or reduce the transcription and/or translation level compared to the unmodified state. Modifications can be single nucleotide modifications or exchange of complete functional elements. In either case the non-natural level of transcription and/or translation is optimized for the transport of the intracellular oligosaccharide of interest across the inner membrane.

In another embodiment the gene or genes encoding the saccharide transporter or transporter complex is/are exogenous to the genetically engineered microbial cell. In such embodiments, the protein coding region is operably linked to naturally occurring or artificial functional elements such as promoters or ribosome binding sites in a way that the transcription level and the translation level of the transporter is optimized for the transport of the oligosaccharide of interest across the inner membrane.

Transcription of the gene(s) encoding the saccharide transporter or the subunits of the saccharide transporter can either be constitutive or regulated. A person skilled in the art would know how to choose and use a promoter and/or a promoter in combination with a further transcriptional regulator to achieve optimal expression of the gene(s) encoding the saccharide transporter.

In embodiments wherein the gene(s) encoding the saccharide transporter is exogenous to the microbial cell for the production of the oligosaccharide of interest, the nucleotide sequence of the gene(s) may be modified as compared to the naturally occurring nucleotide sequence, but may encode a polypeptide possessing an unaltered amino acid sequence.

The saccharide transporter for translocating the oligosaccharide of interest across the cell membrane can have its native amino acid sequence, i.e. the amino acid sequence that is naturally found. Alternatively, the amino acid sequence of the saccharide transporter can be modified as compared to its native amino acid sequence such that the resulting variant has enhanced activity with respect to the translocation of the oligosaccharide of interest and/or may have other beneficial characteristics, e.g. enhanced protein stability or more stable integration into the inner membrane.

In particular embodiments, the nucleotide sequence encoding the saccharide transport protein(s) is artificial and accordingly, the amino acid sequence of the resulting protein(s) acting as transporter to export the oligosaccharide of interest from the cytoplasm into the periplasm is as such not found in nature.

The term “variant(s)” as used herein, refers to a polynucleotide or polypeptide that differs from a reference polynucleotide or polypeptide respectively, but retains the essential (enzymatic) properties of the reference polynucleotide or polypeptide. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques, by direct synthesis, and by other recombinant methods known to the persons skilled in the art.

Within the scope of the present invention, also nucleic acid/polynucleotide and polypeptide polymorphic variants, alleles, mutants, and interspecies homologs are comprised by those terms, that have an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino acid sequence identity, preferably over a region of at least about 25, 50, 100, 200, 500, 1000, or more amino acids, to a polypeptide encoded by a wildtype protein.

A “variant” of any gene/protein is meant to designate sequence variants of the gene/protein which retain the same, a lower or a higher activity of the gene, the protein and/or the protein that is encoded by the gene, or a different activity in regard to the substrate specificity of the protein, or a different activity in regard to the reaction specificity of the protein said variant is derived from.

With respect to the enzymes that are directly involved in the pantothenic acid biosynthesis pathway, a “functional variant” is meant to designate sequence variants of the enzymes and/or the genes encoding said enzymes which enzymes retain the same but lower activity than the respective gene/enzyme said variant is derived from.

The genetically engineered microbial cell for the production of an oligosaccharide of interest lacks an intracellular biosynthesis of pantothenic acid or it possesses a low level of intracelluar pantothenic acid biosynthses.

In some embodiments, the genetically engineered microbial cell for the production of the oligosaccharide of interest is not able to intracellularly synthesize pantothenic acid. In an additional and/or alternative embodiment, the genetically engineered microbial cell has been genetically engineered for being incapable to intracellularly synthesize pantothenic acid, i.e. in that the pantothenic acid biosynthesis pathway within the microbial call has been disrupted.

Disruption of the pantothenic acid biosynthesis pathway can be achieved in that at least one of the microbial cell's genes that encodes an enzyme which is directly involved in pantothenic acid biosynthesis pathway is deleted or functionally inactivated, i.e. in one of the succeeding enzymatic reactions leading to the intracellular formation of pantothenic acid from any given educt. Preferably, the enzyme only catalyzes a reaction that is exclusive to the pantothenic acid biosynthesis pathway.

Said at least one gene that encodes an enzyme which is directly involved in pantothenic acid biosynthesis pathway may be selected from the group of genes which encode a ketopantoate hydroxymethyltransferase, for example panB, a pantothenate synthetase, for example panC, a ketopantoate reductase, for example panE, and an acetohydroxyacid isomeroreductase such as ivfC.

The at least one gene which encodes an enzyme that is directly involved in the pathway of pantothenic acid biosynthesis may be deleted in its entirety or a substantial part of said gene may be deleted. The term “substantial” with respect to the part of the at least one gene refers to nucleotide sequences of said gene that are essential of the expression and/or activity of the enzyme that is encoded by said gene. A “substantial part” of the gene in the meaning of the instant disclosure is understood to include expression control sequences such as the promoter, and/or a protein-coding region of the gene wherein the protein-coding region encodes an amino acid sequence whose presence within the enzyme is essential for the enzymatic activity of the enzyme. Thus, the expression of the gene and/or the enzymatic activity of the polypeptide that is encoded by the gene is abolished.

Functional inactivation of the at least one gene that encodes an enzyme that is directly involved in the biosynthetic pathway of pantothenic acid may be achieved in that at least one codon of the gene is replaced with a codon that encodes a different amino acid than the codon that has been replaced, such that the polypeptide being encoded by the thus amended gene does not possess enzymatic activity for the pantothenic acid biosynthetic pathway. In other embodiments, a codon of the gene that encodes an enzyme that is directly involved in the pantothenic acid biosynthesis pathway is replaced with a stop-codon such that the thus amended gene encodes a fragment of the native enzyme, which fragment does not possess enzymatic activity for the pantothenic acid biosynthetic pathway.

In yet other embodiments, the at least one gene that encodes an enzyme that is directly involved in the biosynthetic pathway of pantothenic acid is functionally inactivated in that a frame shift mutation is inserted into the protein-coding region of said gene such that the polypeptide that is encoded by the thus amended gene does not exhibit enzymatic activity for the pantothenic acid biosynthetic pathway.

Pantothenic acid is the precursor of Coenzyme A. Coenzyme A is an essential compound of a cell's metabolism. In embodiments, wherein the expression or activity of at least one of the enzymes that is involved in the direct pantothenic acid biosynthesis pathway is abolished, the microbial cell can not synthesize pantothenic acid. Such microbial cell requires internalization of exogenous pantothenic acid. Hence, the microbial cell wherein the expression or activity of at least one of the enzymes that is directly involved in the pantothenic acid biosynthesis pathway is abolished possesses a pantothenic acid permease for internalization of exogenous pantothenic acid. Possessing a pantothenic acid permease enables the microbial cell to internalize exogenous pantothenic acid, to proliferate and to produce the oligosaccharide of interest despite its inability to synthesize pantothenic acid.

In some embodiments, the microbial cell for the production of an oligosaccharide of interest which is unable to synthesize pantothenic acid has been genetically engineered to possess and expresses a gene that encodes a pantothenic acid permease.

A suitable pantothenic acid permease is E. coli PanF. Hence, in some embodiments, the microbial cell for the production of the oligosaccharide of interest contains and expresses a gene encoding E. coli PanF or a functional variant thereof, preferably the E. coli panF gene or a functional variant thereof.

Cultivating the microbial cell that is unable to synthesize pantothenic acid, but capable of internalizing exogenous pantothenic acid in the presence of exogenous pantothenic acid is advantageous, because the amount of pantothenic acid in the culture medium can be adjusted. The amount of pantothenic acid in the culture medium can be adjusted such that the microbial cells can proliferate and synthesize the oligosaccharide of interest in versatile amounts, whereas the presence of excessive pantothenic acid in the culture medium at the end of the fermentative production step for the oligosaccharide of interest can be avoided.

In some embodiments, the genetically engineered microbial cell for the production of the oligosaccharide of interest possesses a low level of intracellular pantothenic acid biosynthesis. In some embodiments, the microbial cell has been genetically engineered such that the expression and/or the activity of at least one enzyme being directly involved in the pantothenic acid biosynthesis pathway is impaired.

The term “impaired” as used herein means that the expression and/or activity of the at least one enzyme being directly involved in the pantothenic acid biosynthesis is reduced as compared to the expression and/or activity of said enzyme in microbial cells of the progenitor cell line that were not genetically engineered to possess a reduced and thus low level of pantothenic acid biosynthesis.

The expression “reduced” with respect to the level of intracellular biosynthesis of pantothenic acid is understood as the level of intracellular pantothenic acid biosynthesis as compared to the wild-type microbial cell, i.e. the type of microbial cell of the same genus that is occurring most commonly in nature, especially if the microbial cell is a cell that has not been obtained by genetic engineering to reduce intracellular pantothenic acid biosynthesis, but by e.g. selection with or without preceeding random mutagenesis, or—if the microbial cas been genetically engineered to impair the level of pantothenic acid biosynthesis—as compared to cells of the progenitor cell line that was not yet genetically engineered to possess a reduced intracellular level of pantothenic acid biosynthesis. Apart from the genetic information being engineered to reduce the level of intracellular pantothenic acid biosynthesis, the genetically engineered microbial cell is substantially identical to cells of the progenitor cell line with respect to their genotypes if the levels of intracellular pantothenic acid biosynthesis are to be compared, notwithstanding that the microbial cell as well as cells of their progenitor cell line may be genetically engineered microbial cells that were genetically engineered for other purposes than impairing the intracellular pantothenic acid biosynthesis, e.g. that were engineered for being able to synthesize the oligosaccharide of interest.

The level of pantothenic acid biosynthesis in a genetically engineered microbial cell which possesses an impaired or low pantothenic acid biosynthesis is reduced such that the amount of pantothenic acid in the culture medium is reduced by a factor of at least 10, at least 20, at least 30, at least 40, at least 50, at least 100, at least 200, at least 300, at least 400, or at least 500. It is understood that the reduction of pantothenic acid in the culture medium is assessed in that the cultures of the microbial cell possessing the low level of pantothenic acid biosynthesis and the culture of the microbial cell for comparison were inoculated and incubated at substantially identical conditions, e.g. with respect to growth stage and cell density in the pre-culture, the volume of medium to be inoculated, the medium composition, the amount of pre-culture/number of cells to be used for inoculating the main culture, the cultivating conditions such as temperature, shaking speed, time point/cell density at which samples are taken for measuring the amount of pantothenic acid in the culture medium, etc. Preferably, said reduced level of pantothenic acid in the culture medium is present at the end of the fermentative production of the oligosaccharide of interest.

In some embodiments, the level of pantothenic acid is reduced in that the expression of at least one gene which encodes an enzyme that is directly involved in the biosynthesis pathway of pantothenic acid is reduced. The expression of said gene may be reduced in that the nucleotide sequence of at least one expression control sequence of said gene is altered such that the expression of the protein-coding region for said enzyme is reduced. Examples of altering the nucleotide sequence of an expression control sequence for reducing the expression of the gene that encodes an enzyme that is directly involved in the biosynthesis pathway of pantothenic acid include the deletion of one or more nucleotides, the insertion or the exchange of one or more nucleotides within the expression control sequence of the native gene. Another example of altering the nucleotide sequence of an expression control sequence for reducing the expression of the gene that encodes an enzyme that is directly involved in the biosynthesis pathway of pantothenic acid includes the replacement of a given promoter by another promoter, said another promoter being a weaker promoter than the predecessor promoter. Given that the strength of a promoter is known or can be determined by assays known in the art, it is possible to replace the native promoter of said gene with a heterologous promoter that accounts for a weaker expression of the protein-coding region of the resulting recombinant gene.

In some embodiments, the level of translation, i.e. the level of translating the mRNA into an amino acid sequence, is as compared to the level translation found in the wild type cell or the precursor cell. The level of translation can be modified by changing one or more nucleotides within the ribosome-binding site and/or in the nucleotide sequence up-stream of the ribosome binding site, thereby affecting the translation to be reduce as compared to the level as found in the unmodified state.

In additional and/or alternative embodiments, the microbial cell possesses a gene which encodes a variant of an enzyme that is directly involved in the pantothenic acid biosynthesis pathway, wherein said variant exhibits a reduced enzymatic activity. In some embodiments, the protein-coding region of said gene was modified to encode said variant. In some embodiments, the microbial cell has its endogenous gene deleted or functionally inactivated, and contains a heterologous gene which encodes the variant of said enzyme being directly involved in the biosynthesis pathway of pantothenic acid.

In additional and/or alternative embodiments, the microbial cell possesses a variant of an enzyme that is directly involved in the pantothenic acid biosynthesis pathway which possesses a higher Kₘ (Michaelis constant) value for its substrate (i.e. has a decreased activity) as compared to the native enzyme. The Kₘ value of the variant is preferably increased by at least 100-fold, more preferably by at least 200-fold, and most preferably by at least 500-fold as compared to the Kₘ value of the native enzyme.

The Kₘ value of an enzyme that is directly involved in the pantothenic acid biosynthesis pathway can be increased in that at least one amino acid of said enzyme that is involved in the catalytic activity/substrate binding of the enzyme is replaced with a different amino acid. The amino acid sequence of the enzyme can be amended in that one or more codons of the protein-coding region of the gene encoding said enzyme that is directly involved in the pantothenic acid biosynthesis pathway is replaced with a codon that encodes a different amino acid.

Alternatively, the variant of the enzyme that is directly involved in the pantothenic acid biosynthesis can be modified as compared to the wild-type enzyme by exchanging at least one amino acid of the amino acid sequence which amino acid is involved in the catalytic activity such that the variant has a lower k_{cat}. The k_{cat} value is preferably 10-fold, 100-fold or 500-fold lower as compared to the wild-type enzyme.

In some embodiments is the endogenous gene that encodes the enzyme that is directly involved in the pantothenic acid biosynthesis pathway is altered. In alternative embodiments is the endogenous gene deleted or functionally inactivated, e.g. as described herein before, and the variant of said enzyme is expressed from a recombinant gene.

In particular embodiments, the microbial cell lacks its endogenous ketopantoate reductase, and possesses a ketopantoate reductase which has a Kₘ value for ketopantoate that is at least 100-fold, preferably at least 200-fold, more preferably at least 500-fold higher than the Kₘ value for ketopantoate of the E. coli ketopantoate reductase PanE having the amino acid sequence shown in SEQ ID No. 1. The Michaelis-Menten constant (Kₘ) is defined as the substrate concentration at which the reaction rate is half of its maximum value (or in other words it defines the substrate concentration at which half of the active sites are occupied).

In some embodiments, the ketopantoate reductase possessing a higher Kₘ value for ketopantoate than the E. coli ketopanthotenate reductase having the amino acid sequence shown in SEQ ID No. 1 is a variant of E. coli PanE which possesses an increased Kₘ value for ketopantothenate. The Kₘ value is preferably increased at least 100-fold, more preferably at least 200-fold, and most preferably at least 500-fold.

In an additional and/or alternative embodiment, the ketopantoate reductase possessing an increased Kₘ value for ketopantoate is a variant of the E. coli K-12 PanE enzyme which possesses an amino acid comprising a pure hydrocarbon side chain at position 98,176, 244 and/or 256 with respect to the amino acid sequence shown in SEQ ID No. 1. The amino acid comprising a pure hydrocarbon side chain is an amino acid selected from the group consisting of glycine (Gly, G), alanine (Ala, A), valine (Val, V), leucine (Leu, L), isoleucine (Ile, I), phenylalanine (Phe, F) and proline (Pro, P).

In particular embodiments, the variants of the ketopantoate reductase which possess an increased Kₘ value for ketopantoate are selected from the group of variants consisting of E. coli K-12 PanE (N98A), E. coli K-12 PanE (K176A), E. coli K-12 PanE (S244A), E. coli K-12 PanE (E256A), and combinations of said variations with the provision of the variant E. coli K-12 (K176A, E256A) and other variants which lack ketopantoate reductase activity. The increase of the Kₘ value for ketopantoate caused by the different variations are shown in Table 1.

TABLE 1
Increase of Kₘ value for ketopantoate in different variants
of the E. coli K-12 ketopantoate reductase PanE.

| | | X-fold increase of |
|---|---|---|
| Position | Variant | Kₘ for ketopantoate |
| 98 | N → A | 140 |
| 176 | K → A | 1400 |
| 244 | S → A | 230 |
| 256 | E → A | 1100 |

Microbial cells possessing a low level of intracellular pantothenic acid biosynthesis are advantageous for use in the production of an oligosaccharide of interest, because these microbial cells can synthesize pantothenic acid intracellularly and therefore do not require supply of exogenous pantothenic acid when cultured, while the amount of pantothenic acid in the culture medium at the end of the fermentation step for the production of the oligosaccharide of interest is low. Said low level of pantothenic acid in the culture medium allows to facilitate recovery of the oligosaccharide of interest.

According to the second aspect, provided is the use of a genetically engineered microbial cell possessing a low level of or no pantothenic acid biosynthesis as disclosed herein before for the production of an oligosaccharide of interest. The genetically engineered microbial cells synthesize said oligosaccharide of interest when they are cultured in a medium and under conditions that are permissive for the microbial cell to synthesize the oligosaccharide of interest.

Utilizing these genetically engineered microbial cells for the production of an oligosaccharide of interest is advantageous over the use of microbial cells that do not possess a low level of pantothenic acid biosynthesis, because the amount of pantothenic acid in the fermentation broth at the end of the culturing step is much lower. Preferably, the amount of pantothenic acid in the fermentation broth at the end of the fermentation step is so low that the effort in the subsequent purification of the oligosaccharide of interest from the fermentation broth does not require one or more process steps that are tailored to remove pantothenic acid from the fermentation broth or process stream obtained from the fermentation broth.

It has surprisingly been found, that the genetically engineered microbial cells possessing a low level of pantothenic acid biosynthesis, display a higher production of the oligosaccharide of interest as compered to cells of their progenitor cell line that were genetically engineered to possess a low level of pantothenic acid biosynthesis.

In some embodiments, the production of the oligosaccharide of interest is increased by at least 10%, at least 15%, at least 20%, at least 25%, at least 30%, at least 35%, or at least 40% as compared to the production of the oligosaccharide of interest by cells of their progenitor cell line that were not genetically engineered to possess a low level of pantothenic acid biosynthesis.

According to the third aspect, provided is a method for producing an oligosaccharide of interest in a genetically engineered microbial cell, the method comprises
providing a genetically engineered microbial cell which possesses a metabolic pathway for intracellular synthesis of the oligosaccharide of interest, and a low level of or no pantothenic acid biosynthesis;
culturing the genetically engineered microbial cell in a medium and at conditions that are permissive for the microbial cell to synthesize the oligosaccharide of interest intracellularly; and
optionally recovering the oligosaccharide of interest.

The genetically engineered microbial cell which possesses a metabolic pathway for intracellular synthesis of the oligosaccharide of interest, and a low level of or no pantothenic acid biosynthesis is preferably a microbial cell as described herein before.

The oligosaccharide of interest is an oligosaccharide as described herein before, preferably a HMO.

In some embodiments, preferably in embodiments wherein the genetically engineered microbial cell does not possess a pantothenic acid biosynthesis, the genetically engineered microbial cell is cultured in the presence of exogenous pantothenic acid. That said, it is understood that pantothenic acid is added to the culture medium before and/or during culturing the genetically engineered microbial cell. The supply of exogenous pantothenic acid to the culture medium can be adjusted according to predetermined process parameters and/or given requirements. The supply of exogenous pantothenic acid to the culture medium is ceased prior to the end of the fermentative production step for producing the oligosaccharide of interest such that the culture medium contains less than 500 μg/ml, 400 μg/ml, 300 μg/ml, 200 μg/ml, 100 μg/ml, 80 μg/ml, 60 μg/ml, or even less than 40 μg/ml at the end of the fermentation step for having the oligosaccharide of interest synthesized.

According to the fourth aspect, provided is a preparation of an oligosaccharide of interest, preferably a HMO as described herein before, wherein said preparation is obtained by using a microbial cell or by employing a method for producing the oligosaccharide of interest as disclosed herein before.

Said preparation of the oligosaccharide of interest may be used for the manufacturing of a nutritional composition. The nutritional composition may be a nutritional supplement, a medicinal food, an infant formula, or any other food being limited with respect to the maximum amount of pantothenic acid contained therein. Said nutritional composition preferably contains the oligosaccharide of interest in an amount substantially similar to the amount of the oligosaccharide of interest naturally present in human milk. Using said preparation of the oligosaccharide of interest for the manufacturing of a nutritional composition, especially an infant formula, is advantageous, because the risk of exceeding the upper limit of pantothenic acid prescribed for such nutritional composition is mitigated, and neither costly process steps for the removal of pantothenic acid in the course of the production of the oligosaccharide of interest nor adjustments of an already existing production process for the nutritional composition are required.

The present invention also extends to the nutritional compositions which contain one or more oligosaccharides of interest that were produced by using the genetically engineered microbial cells and/or the method for producing the oligosaccharides of interest being described herein before.

The present invention will be described with respect to particular embodiments and with reference to drawings, but the invention is not limited thereto but only by the claims. Furthermore, the terms first, second and the like in the description and in the claims, are used for distinguishing between similar elements and not necessarily for describing a sequence, either temporally, spatially, in ranking or in any other manner. It is to be understood that the terms so used are interchangeable under appropriate circumstances and that the embodiments of the invention described herein are capable of operation in other sequences than described or illustrated herein.

It is to be noticed that the term “comprising”, used in the claims, should not be interpreted as being restricted to the means listed thereafter; it does not exclude other elements or steps. It is thus to be interpreted as specifying the presence of the stated features, integers, steps or components as referred to, but does not preclude the presence or addition of one or more other features, integers, steps or components, or groups thereof. Thus, the scope of the expression “a device comprising means A and B” should not be limited to devices consisting only of components A and B. It means that with respect to the present invention, the only relevant components of the device are A and B.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

Similarly, it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the detailed description are hereby expressly incorporated into this detailed description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

Furthermore, some of the embodiments are described herein as a method or combination of elements of a method that can be implemented by a processor of a computer system or by other means of carrying out the function. Thus, a processor with the necessary instructions for carrying out such a method or element of a method forms a means for carrying out the method or element of a method. Furthermore, an element described herein of an apparatus embodiment is an example of a means for carrying out the function performed by the element for the purpose of carrying out the invention.

In the description and drawings provided herein, numerous specific details are set forth. However, it is understood that embodiments of the invention may be practiced without these specific details. In other instances, well-known methods, structures and techniques have not been shown in detail in order not to obscure an understanding of this description.

The invention will now be described by a detailed description of several embodiments of the invention. It is clear that other embodiments of the invention can be configured according to the knowledge of persons skilled in the art without departing from the true spirit or technical teaching of the invention, the invention being limited only by the terms of the appended claims.

### EXAMPLES

### LC/MS Analytics

Product identity was determined by multiple reaction monitoring (MRM) using an LC triple-quadrupole MS detection system (Shimadzu LCMS-8050). Precursor ions were selected and analyzed in quadrupole 1, followed by collision-induced fragmentation (CID) with argon and selection of fragment ions in quadrupole 3. Selected transitions and collision energies for intermediates and end-product metabolites are listed in Table 2. LNT, 3′-SL and pantothenic acid (Vitamin B5) in particle free culture supernatant or cytoplasmic fractions were separated by high performance liquid chromatography (HPLC) using a Waters XBridge BEH Amide HPLC column (3.5 μm, 2.1×50 mm) protected by a XBridge BEH Amide VanGuard cartridge (3.9×5 mm) for both neutral or acidic sugars. Neutral sugars were eluted with isocratic flow of H₂O with 0.1% (v/v) ammonium hydroxide. Before LC/MS analysis, the samples of neutral oligosaccharides were prepared by filtration (0.22 μm pore size) and cleared by solid-phase extraction on a Strata ABW ion exchange matrix (Phenomenex). 3′-Sialyllactose and pantothenic acid containing samples were filtered through a 0.22 μm filter, heated for 5 min at 95° C. and centrifuged for 5 min at 13.000×g. HPLC separation of the acidic sugar was performed using gradient elution with buffer A consisting of 50% acetonitrile and buffer B consisting of 80% acetonitrile, both containing 10 mM ammonium acetate at pH 4.2. The HPLC system encompasses a Shimadzu Nexera X2 SIL-30AC_{MP} autosampler run at 8° C., a Shimadzu LC-20AD pump, and a Shimadzu CTO-20AC column oven running at 35° C. for elution of neutral sugars and was adjusted to 50° C. for elution of acidic sugars. For LNT analysis 1 μl, for 3′-SL and pantothenic acid determination 0.5 μL was injected into the instrument. Flow rate was set 0.3 mL/min (neutral), 0.4 mL/min (acidic) corresponding to a run time of 5 min. Whereas LNT was analyzed in negative ion mode, 3′-SL and pantothenic acid were examined in positive ion mode. The mass spectrometer was operated at unit resolution. Collision energy, Q1 and Q3 pre-bias were optimized for each analyte. Quantification methods were established using commercially available standards (Carbosynth, Elicityl, Sigma Aldrich).

TABLE 2
List of diagnostic MRM transitions and parameters for
the identification of LNT, 3′-SL and pantothenic
acid (Vitamin B5) (CE = Collision energy) of the
Shimadzu LCMS-8050 Triple Quadrupole (QQQ) Mass Analyzer

| | | Pre- | | Dwell | Q1 pre | | Q3 pre |
|---|---|---|---|---|---|---|---|
| | | cursor | Product | time | bias | | bias |
| Metabolite | Mode | m/z | m/z | [msec] | (V) | CE | (V) |
| 3′-SL | + | 656.2 | 365.15 | 59 | −22 | −33 | −27 |
| | + | 656.2 | 314.2 | 59 | −22 | −33 | −16 |
| Panto- | + | 219.9 | 90.1 | 100 | −11 | −16 | −15 |
| thenic | | | | | | | |
| acid | | | | | | | |
| | + | 219.9 | 98.05 | 100 | −11 | −23 | −16 |
| | + | 219.9 | 202.15 | 100 | −11 | −14 | −20 |
| LNT | − | 706.2 | 202.1 | 9 | 32 | 22 | 19 |
| | − | 706.2 | 142.0 | 9 | 32 | 31 | 23 |
| | − | 706.2 | 382.1 | 9 | 32 | 17 | 26 |

### Culture Medium

The culture medium used to grow the cells for the production of the desired oligosaccharide contained: 3 g/L KH₂PO₄, 12 g/L K₂HPO₄, 5 g/L (NH₄)SO₄, 0.3 g/L citric acid, 0.1 g/L NaCl, 2 g/L MgSO₄×7 H₂O and 0.015 g/L CaCl₂×6H₂O, supplemented with 1 ml/L trace element solution (54.4 g/L ammonium ferric citrate, 9.8 g/L MnCl₂×4 H₂O, 1.6 g/L CoCL₂×6 H₂O, 1 g/L CuCl₂×2 H₂O, 1.9 g/L MnCl₂×4 H₂O, 1.1 g/L Na₂MoO₄×2 H₂O, 1.5 g/L Na₂SeO₃, 1.5 g/L NiSO₄×6 H₂O). The pH of the medium was adjusted to 7.0. For synthesis of the desired oligosaccharide lactose was added to the medium.

### Example 1: Metabolic Engineering of E. coli to Reduce the Pantothenic Acid Biosynthesis

To introduce the mutations into the panE gene resulting in the amino acid exchange S244A within E. coli PanE, an antibiotic resistance cassette was inserted between genes thiI and yjaL by homologous recombination. The antibiotic resistance cassette was flanked by 640 nucleotides representing the 3′-end of thiI and 777 nucleotides comprising yjaL and the 3′-end of panE. The flanking regions were amplified from genomic DNA of E. coli BL21 (DE3). While amplifying the yjaL-'panE flank two single point mutation were introduced resulting in the amino acid exchange serine to alanine at position 244 in the PanE protein. Homologous recombination was performed and facilitated by expression of lambda red genes gam, beta, and exo from plasmid pKD46 according to Datsenko and Wanner (Proc. Natl. Acad. Sci. (2000) 97, 6640-66645 (2000)). Mutations were confirmed by sequencing the panE gene in the E. coli genome.

### Example 2: Production of Sialyllactose with Decreased Pantothenic Acid

A metabolically engineered derivative of E. coli BL21(DE3) being able to synthesize 3′-sialyllactose intracellularly was genetically engineered to reduce pantothenic acid biosynthesis.

The strain was grown in mineral salts medium using glycerol as sole carbon source. A pre-culture of 200 ml was inoculated and grown for 24 h at 30° C. under aerobic conditions. This pre-culture was used to inoculate a fermenter containing 1 L of mineral salts medium and 2% glycerol to an optical density of (OD₆₀₀=0.15. Fermentation was conducted at 30° C. and pH 7.0 under aerobic conditions. 100 mM lactose were fed to the culture before the glycerol was consumed starting at an OD₆₀₀ of about 5. When entering the fed-batch phase glycerol was fed continuously and cells were grown under limitation of the C-source. Concentration of 3′-SL and pantothenate was measured in the culture supernatant.

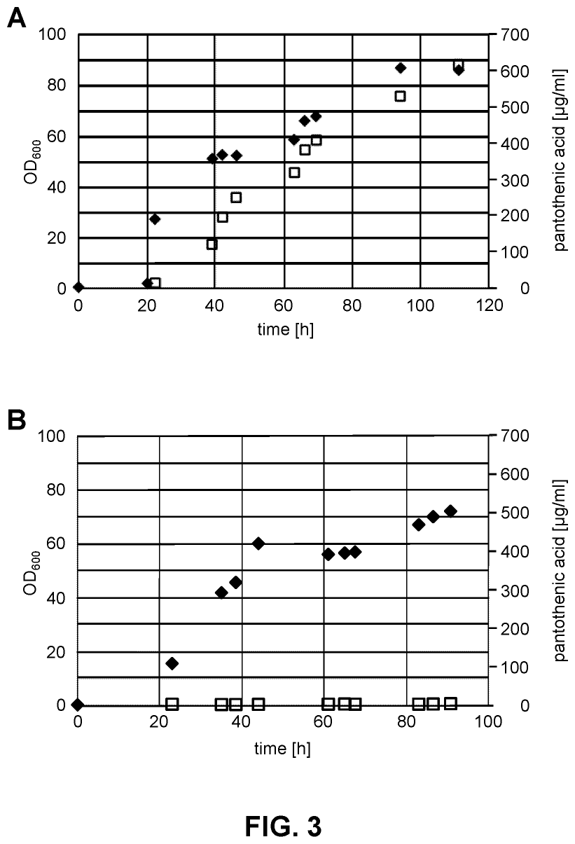

FIG. 3 displays the growth (♦) of the bacterial cell cultures and the amount of pantothenic acid (□) in the culture medium of the bacterial cells, wherein the graph of FIG. 3 A illustrates the growth and pantothenic acid amount of the parental E. coli strain, whereas the graph of FIG. 3 B illustrates the growth and pantothenic acid amount of the E. coli strain that was genetically engineered to possess a reduced, i.e. low level of pantothenic acid biosynthesis.

After 95 h of fermentation a 3′-SL titer of was achieved that was 1.15 times higher using the E. coli strain that was genetically engineered to possess a reduced level of pantothenic acid biosynthesis than the 3′-SL titer of the parental strain grown at the same conditions. In addition, the level of pantothenic acid in the culture supernatant was 0.01-fold of the pantothenic acid level in the culture supernatant of the parental strain that grew at the same conditions.

### Example 3 Production of Lacto-N-Tetraose with Decreased Pantothenic Acid

A genetically engineered derivative of the E. coli strain BL21 (DE3) which is able to synthesize lacto-N-tetraose intracellularly was further engineered to possess reduced pantothenic acid biosynthesis. The derivative's endogenous panE gene was replaced with a variant which encodes PanE S244A.

The latter LNT-producing E. coli strain was grown in mineral salts medium using glycerol as sole carbon source. Four pre-cultures of 200 ml were inoculated and grown for 40 h at 30° C. under aerobic conditions. These pre-cultures were used to inoculate a seed fermenter containing 180 L of the mineral salts medium to an optical density of OD₆₀₀=0.1. This well growing seed culture was used to inoculate the production fermenter containing 2500 L of the mineral salts medium supplemented with 60 mM lactose, 5 g/L NH₄Cl and 2% glycerol to an optical density of OD₆₀₀=0.1. Culturing of the cells was conducted at 30° C. and pH 6.8 under aerobic conditions. Glycerol was fed continuously when the culture entered the fed-batch phase, and the bacteria were further grown under limitation of the C-source. Upon stabilized dissolved oxygen level, a continuous lactose feed was initiated in order to keep lactose concentration in the medium at a sufficient level for LNT production. Concentration of LNT and pantothenate was measured in the culture supernatant.

After 86 h of fermentation the concentration of pantothenic acid in the culture supernatant was more than 16-fold lower as compared to the supernatant of a cell culture of the parental strain. Surprisingly, the amount of LNT in the culture supernatant was increased by more than 1.4-fold in the culture of the E. coli strain possessing the diminished pantothenic acid biosynthesis as compared to the parental strain.

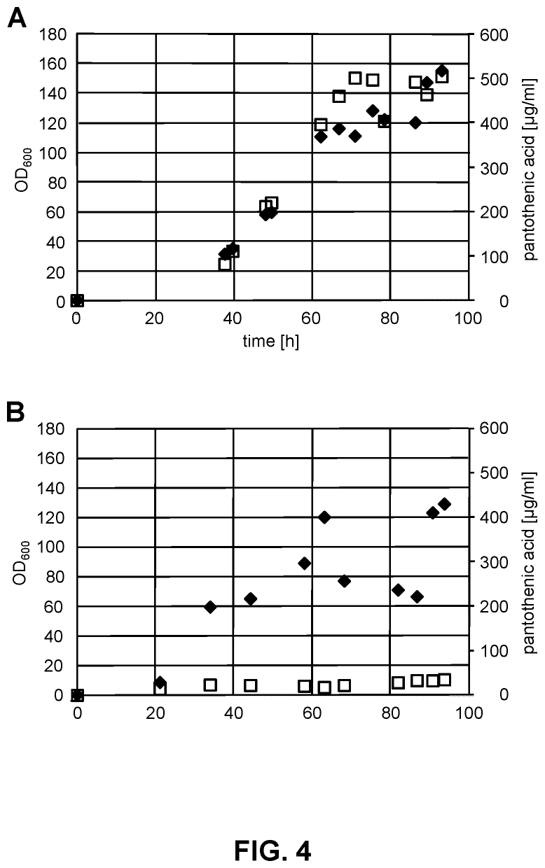

FIG. 4 displays the growth (♦) of the bacterial cell cultures and the amount of pantothenic acid (□) in the culture medium of the bacterial cells, wherein the graph of FIG. 4 A illustrates the growth and pantothenic acid amount of the parental E. coli strain, whereas the graph of FIG. 4 B illustrates the growth and pantothenic acid amount of the E. coli strain that was genetically engineered to possess a reduced, i.e. low level of pantothenic acid biosynthesis.

1. A genetically engineered microbial cell for the production of an oligosaccharide of interest, wherein said microbial cell possess a metabolic pathway for the intracellular biosynthesis of the oligosaccharide of interest, and a low level of or no intracellular biosynthesis of pantothenic acid.

2. The genetically engineered microbial cell according to claim 1, wherein the oligosaccharide of interest is selected from the group of Human Milk Oligosaccharides, preferably from the group consisting of 2′-fucosyllactose (2′-FL), 3-fucosyllactose (3-FL), 2′,3-difucosyllactose (DFL), lacto-N-triose II, lacto-N-tetraose (LNT), lacto-N-neotetraose (LNnT), lacto-N-fucopentaose I (LNFP-I), lacto-N-neofucopentaose I (LNnFP-I), lacto-N-fucopentaose II (LNFP-II), lacto-N-fucopentaose III (LNFP-III), lacto-N-fucopentaose V (LNFP-V), lacto-N-neofucopentaose V (LNnFP-V), lacto-N-hexaose (LNH), lacto-N-neohexaose (LNnH), para-lacto-N-hexaose (paraLNH), para-lacto-N-neohexaose (paraLNnH), difucosyl-lacto-N-neohexaose (DF-LNnH), lacto-N-difucosylhexaose I, lacto-N-difucosylhexaose II, para-lacto-N-fucosylhexaose (paraLNH), fucosyl-lacto-N-sialylpentaose a (F-LST-a), fucosyl-lacto-N-sialylpentaose b (F-LST-b), fucosyl-lacto-N-sialylpentaose c (F-LST-c), fucosyl-lacto-N-sialylpentaose c, disialyl-lacto-N-fucopentaose, 3-fucosyl-3′-sialyllactose (3F-3′-SL), 3-fucosyl-6′-sialyllactose (3F-6′-SL), lacto-N-neodifucohexaose I, 3′-sialyllactose (3′-SL), 6′-sialyllactose (6′-SL), sialyllacto-N-tetraose a (LST-a), sialyllacto-N-tetraose b (LST-b), sialyllacto-N-tetraose c (LST-c), disialyllacto-N-tetraose (DS-LNT), Disialyl-lacto-N-fucopentaose (DS-LNFP V), lacto-N-neodifucohxaose I (LNnDFH I), 3′-galactosyllactose (3′-GL), 6′-galactosyllactose (6′-GL).

3. The genetically engineered microbial cell according to claim 1, wherein an endogenous gene encoding an enzyme that is directly involved in the pantothenic acid biosynthesis pathway has been deleted or functionally inactivated.

4. The genetically engineered microbial cell according to claim 1, wherein the low level of pantothenic acid biosynthesis leads to a reduced amount of pantothenic acid in the culture medium by a factor of at least 10.

5. The genetically engineered microbial cell according to claim 1, wherein the expression and/or activity of at least one enzyme directly involved in the intracellular pantothenic acid biosynthesis pathway is impaired.

6. The genetically engineered microbial cell according to claim 1, wherein the at least one enzyme is selected from the group consisting of ketopantoate hydroxymethyltransferase, ketopantoate reductase, acetohydroxy acid isomeroreductase, aspartate 1-decarboxylase and pantothenate synthetase.

7. The genetically engineered microbial cell according to claim 6, wherein the ketopantoate reductase possesses a Kₘ value for ketopantoate that is increase as compared to the E. coli K-12 ketopantoate reductase, preferably at least 100-fold, more preferably at least 200-fold, most preferably at least 500-fold.

8. The genetically engineered microbial cell according to claim 6, wherein ketopantoate reductase is a variant of the E. coli K-12 ketopantoate reductase which variant is selected from the group consisting of E. coli K-12 PanE (N98A), E. coli K12 PanE (K176A), E. coli K-12 PanE (S244A), E. coli K-12 PanE (E256A), and combinations of said variations.

9. (canceled)

10. A method for producing an oligosaccharide of interest, the method comprises
providing a genetically engineered microbial cell which possesses a metabolic pathway for the intracellular biosynthesis of the oligosaccharide of interest, and a low level of or no intracellular biosynthesis of pantothenic acid;
culturing the genetically engineered microbial cell in a medium and under conditions that are permissive for the intracellular biosynthesis of the oligosaccharide of interest; and
optionally retrieving the oligosaccharide of interest.

11. The method according to claim 10, wherein the genetically engineered microbial cell is cultured in the presence of exogenous pantothenic acid.

12. (canceled)

13. A nutritional composition comprising an oligosaccharide of interest, the oligosaccharide produced by a method of claim 10.

14. The nutritional composition of claim 13, wherein the nutritional composition is an infant formula.
